(19) United States
(12) Patent Application Publication
(10) Pub. No.: US 2013/0237800 A1
(43) Pub. Date: Sep. 12, 2013

(54) OBJECT INFORMATION ACQUIRING APPARATUS

(71) Applicant: CANON KABUSHIKI KAISHA, Tokyo (JP)

(72) Inventor: Takeshi Yamamoto, Tokyo (JP)

(73) Assignee: CANON KABUSHIKI KAISHA, Tokyo (JP)

(21) Appl. No.: 13/778,705

(22) Filed: Feb. 27, 2013

(30) Foreign Application Priority Data
Mar. 8, 2012 (JP) 2012-051617

(51) Int. Cl. A61B 5/00

(52) U.S. Cl. CPC A61B 5/0095; A61B 5/708
USPC 600/407

(57) ABSTRACT

The present invention employs an object information acquiring apparatus that includes a light source, a holding unit configured to hold an object, an acoustic receiving unit configured to receive an acoustic wave generated by irradiating light, which is emitted from the light source, on the object via the holding unit and convert the acoustic wave into an electric signal, a light measuring unit configured to measure the light emitted from the light source, a processing unit configured to calculate a light density distribution in the object based on the light measured by the light measuring unit, and generate property information of the inside of the object on the basis of the light density distribution and the electric signal, and a supporting unit configured to support the holding unit and at least a part of the light measuring unit to be removable independently from each other.

### BACKGROUND OF THE INVENTION

1. Field of the Invention

The present invention relates to an object information acquiring apparatus.

2. Description of the Related Art

Researches for a photoacoustic imaging apparatus that irradiates light on a living organism from a light source such as a laser and converts information concerning the inside of the living organism obtained on the basis of incident light into an image have been actively conducted in the medical field. As one of such imaging techniques, photoacoustic tomography (PAT) has been proposed.

The PAT is a technique for irradiating pulsed light generated from a light source on a living organism (an object), receiving an acoustic wave generated from a biological tissue that has absorbed light propagating and diffusing in the living organism, and subjecting the received acoustic wave to analysis processing to visualize information concerning the inside of the living organism, which is the object. According to the technique, it is possible to obtain an optical property distribution, in particular, an optical energy absorption density distribution in the living organism. Researches for diagnosing the object using the optical energy absorption density distribution have been conducted.

According to Japanese Patent Application Laid-Open No. 2011-229756 (Patent Literature 1), in the PAT, initial sound pressure P₀ of an acoustic wave generated from a light absorber in an object can be represented by the following Expression (1):

P₀=Γ·μₐ·Φ (1)

where, Γ represents a Gruneisen coefficient, which is obtained by dividing a product of a coefficient of volume expansion β and a square of sound speed c by a specific heat at constant pressure CP, μₐ represents a light absorption coefficient of the light absorber, and Φ represents a luminous flux.

It is known that the Gruneisen coefficient takes a substantially fixed value if an object is determined. The luminous flux indicates an amount of light in a local region, i.e., an amount of light irradiated on the light absorber. The luminous flux is referred to as optical fluence as well.

A temporal change of sound pressure P, which is the magnitude of the acoustic wave propagating through the object is measured and an initial sound pressure distribution is calculated from a result of the measurement. The calculated initial sound pressure distribution is divided by the Gruneisen coefficient Γ, whereby an optical energy absorption density distribution, which is a product of μₐ and Φ, can be obtained.

As indicated by Expression (1), in order to obtain the distribution of the light absorption coefficient μₐ from the distribution of the initial sound pressure P₀, it is necessary to calculate the distribution of the luminous flux Φ (a light amount distribution) in the object.

According to Japanese Patent Application Laid-Open No. 2011-229756, the distribution of the luminous flux Φ can be calculated using a relative light irradiation density distribution (hereinafter referred to as “relative illuminance distribution”) of light irradiated on the surface of the object. The relative illuminance distribution means a relative light intensity distribution in a light irradiation region on the surface of the object. The relative illuminance distribution can be calculated by imaging an optical pattern generated on the object surface when light is irradiated thereon. It is possible to calculate a light amount distribution in the object by analyzing the relative illuminance distribution. It is possible to obtain a light absorption distribution in the object using the light amount distribution according to Expression (1).

Patent Literature 1: Japanese Patent Application Laid-Open No. 2011-229756

### SUMMARY OF THE INVENTION

In an imaging apparatus that photographs a human or an animal such as X-ray, CT, and MRI apparatuses including a photoacoustic imaging apparatus in the present invention, a method of holding and fixing an object is adopted in order to obtain a clearer image. Therefore, holding instruments having shapes suitable for the shape of an object and an imaging purpose are developed. For example, in commercially available mammography, there are variations in the size of a pressuring plate functioning as a holding instrument. The pressuring plate can be replaced according to the size of a breast. As a result, an operator can easily adjust the shape of the breast during photographing and obtain a desired image. Further, pressuring plates for performing local photographing and enlarged photographing are prepared.

In the apparatus described in Japanese Patent Application Laid-Open No. 2011-229756, similarly, there is a mechanism for holding an object not to move. However, in this apparatus, a light diffusing member for calculating a relative illuminance distribution is attached to a part of a holding unit that comes into contact with the object. Therefore, when the holding unit is replaced, the light diffusing member also has to be attached again. As a result, labor and time are required in replacing the holding unit compared with general mammography or the like.

In order to solve the problem, a measure for using a holding plate attached with a light diffusing member is conceivable. However, in this measure, the area of the light diffusing member is necessary in addition to an area for imaging the object. Therefore, the holding plate is increased in size and complicated in shape, leading to an increase in costs and deterioration in rigidity.

Further, in Japanese Patent Application Laid-Open No. 2011-229756, as a method of calculating a light irradiation density distribution, light irradiated on the light diffusing member is imaged to calculate relative light irradiation density and an amount of the light is separately measured by a light-amount measuring unit to calculate the light irradiation density distribution. However, there is no reference to other means for calculating the light irradiation density distribution.

The present invention has been devised in view of the problems and it is an object of the present invention to, when an apparatus that performs photoacoustic measurement includes a member for holding an object and a member for measuring light, simplify the mechanism of the apparatus and improve easiness of replacement of the members.

The present invention provides an object information acquiring apparatus comprising:

a light source;

a holding unit configured to hold an object;

an acoustic receiving unit configured to receive an acoustic wave generated by irradiating light, which is emitted from the light source, on the object via the holding unit and convert the acoustic wave into an electric signal;

a light measuring unit configured to measure the light emitted from the light source;

a processing unit configured to calculate a light density distribution in the object based on the light measured by the light measuring unit, and generate property information of an inside of the object on the basis of the light density distribution and the electric signal; and

a supporting unit configured to support the holding unit and at least a part of the light measuring unit to be removable independently from each other.

According to the present invention, when an apparatus that performs photoacoustic measurement includes a member for holding an object and a member for measuring light (e.g., a light distribution), it is possible to simplify the mechanism of the apparatus and improve easiness of replacement of the members.

Further features of the present invention will become apparent from the following description of exemplary embodiments with reference to the attached drawings.

### BRIEF DESCRIPTION OF THE DRAWINGS

FIG. 1A is a schematic diagram showing an example of the configuration of an apparatus according to a first embodiment;

FIG. 1B is another schematic diagram showing an example of the configuration of the apparatus according to the first embodiment;

FIG. 2 is a flowchart for explaining an example of the operation of the apparatus according to the first embodiment;

FIGS. 3A and 3B are schematic diagrams showing an example of an opening section of the apparatus according to the first embodiment;

FIG. 4 is a flowchart for explaining calculation of a light irradiation density distribution of the apparatus according to the first embodiment;

FIG. 5 is a schematic diagram showing an example of the configuration of an apparatus according to a second embodiment; and

FIG. 6 is a flowchart for explaining an example of the operation of the apparatus according to the second embodiment.

### DESCRIPTION OF THE EMBODIMENTS

Preferred embodiments of the present invention are explained below with reference to the drawings. However, dimensions, materials, and shapes of components explained below, relative arrangement of the components, and the like should be changed as appropriate according to the configuration of an apparatus to which the present invention is applied and various conditions and are not meant to limit the scope of the present invention to the below description.

In the present invention, an acoustic wave includes waves called sound wave, ultrasound wave, and photoacoustic wave and indicates an elastic wave generated on the inside of an object when light (an electromagnetic wave) such as a near infrared ray is irradiated on the object. A photoacoustic imaging apparatus according to the present invention is an apparatus that acquires property information of the inside of an object and generates image data mainly for diagnosis of malignant tumors, vascular diseases, and the like of a human and an animal, follow-up of a chemical treatment, and the like. Therefore, as the object, a living organism, specifically, a target region of diagnosis such as a breast, a finger, or a limb of a human body or an animal is assumed. A light absorber on the inside of the object relatively has a high absorption coefficient in the object. For example, when a human body is a measurement target, oxygenated or reduced hemoglobin, a vessel including a large quantity of the hemoglobin, or a malignant tumor including a large number of newborn blood vessels corresponds to the light absorber. Since the property information of the inside of the object is also called as object information, the photoacoustic imaging apparatus according to the present invention that acquires such property information can also be called as object information acquiring apparatus.

<First Embodiment>

(Apparatus configuration)

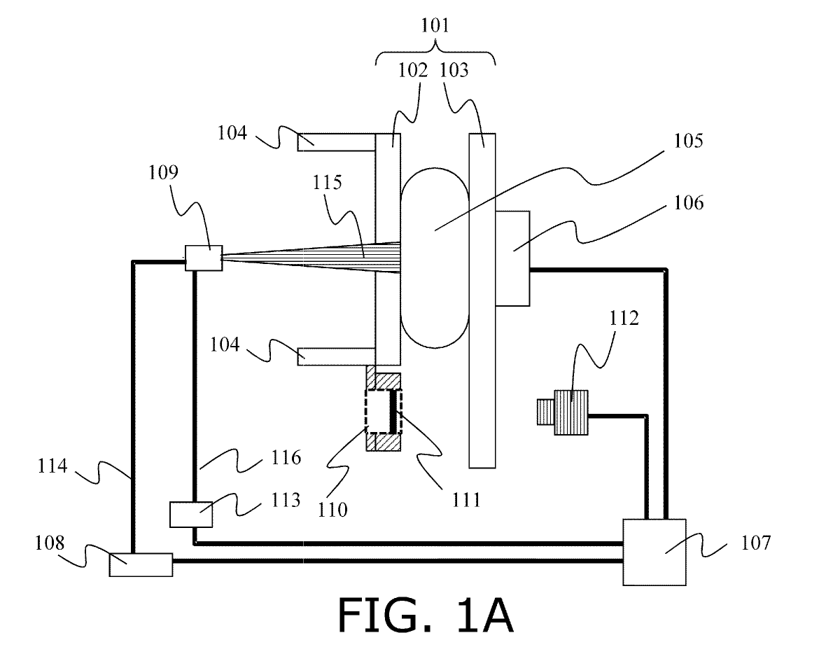

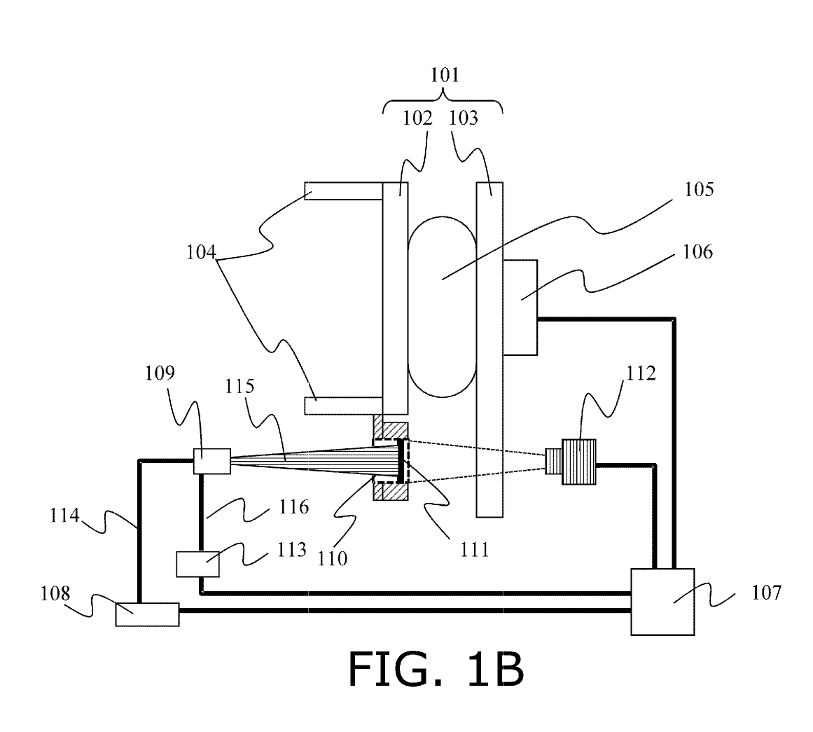

FIG. 1A is a schematic diagram showing the configuration of a photoacoustic imaging apparatus according to a first embodiment. The photoacoustic imaging apparatus according to this embodiment includes a holding unit 101 that holds an object 105 and an optical system 109 that irradiates light on the held object 105. Further, the photoacoustic imaging apparatus includes an acoustic-wave converting unit 106 that receives an acoustic wave generated by irradiation of light and converts the acoustic wave into an electric signal and a processing unit 107 that generates image data from the electric signal.

(Principle of PAT measurement)

PAT measurement performed using the photoacoustic imaging apparatus according to this embodiment is explained. Light 114 emitted from a light source 108 is irradiated on the object 105 such as a living organism via the optical system 109 such as a lens, a mirror, or an optical fiber. The optical system 109 desirably includes a magnifying lens that magnifies an irradiation region of the light 114. The light 114 is changed to irradiation light 115 by the magnifying lens and irradiated on the object 105.

When a part of energy of light propagated through the inside of the object 105 is absorbed by a light absorber (which resultantly becomes a sound source) such as the inside of a blood vessel or the inside of blood, an acoustic wave (typically, an ultrasound wave) is generated by thermal expansion of the light absorber. The acoustic wave is received by the acoustic-wave converting unit 106 and converted into an electric signal. The processing unit 107 generates a light absorption coefficient distribution in the object 105 as image data using an initial sound pressure distribution in the object 105 and a light amount distribution in the object 105 obtained from the electric signal. The image data is displayed as an image on a display device (not shown in the figure) such as a liquid crystal display.

Besides, the apparatus includes, as mechanisms for calculating a light irradiation density distribution, a light diffusing member 111, an opening section 110 to which the light diffusing member 111 is attached, a light-amount measuring unit 113 that receives reference light 116 branched from measurement light in the optical system 109, and an imaging unit 112. Light measurement performed using these mechanisms is explained below.

(Configuration of a holding mechanism)

The holding unit 101 is configured by two holding plates, i.e., a light-irradiation-side holding plate 102 on a side where light is irradiated and a reception-side holding plate 103 on a side where an acoustic wave is received. The light-irradiation-side holding plate 102 is detachably attached to a light-irradiation-side-holding-plate supporting member 104. The light-irradiation-side holding plate 102 and the light-irradiation-side-holding-plate supporting member 104 is configured to be movable in association with each other in order to hold the object 105 in a predetermined position. The object is sandwiched by a pair of plate-like members. However, the object may be pressed against the holding plates and held. The shape of the holding plates is not always limited to the plate-like members.

As the light-irradiation-side holding plate 102, a material having high transparency such as acrylic resin is desirable. As the reception-side holding plate 103, a material having acoustic impedance close to the acoustic impedance of a human breast or the like such as polymethyl pentene polymer is desirable.

As the light-irradiation-side holding plate 102, holding plates having a plurality of shapes that can be replaced according to the shape of the object 105 and a photographing purpose are prepared as variations. The light-irradiation-side holding plate 102 is removably fixed to the light-irradiation-side-holding-plate supporting member 104 according to operation from the object side. The structure of an attaching section and an attaching method only have to be capable of withstanding a load from the object direction when the object is held. However, structure that can be replaced by one hand without using a tool is desirable.

The light diffusing member 111 is fixed to the light-irradiation-side-holding-plate supporting member 104 to keep a fixed distance from a surface of the light-irradiation-side holding plate 102 that is in contact with the object 105. The light diffusing member 111 is fixed to the light-irradiation-side-holding-plate supporting member 104 to be capable of being attached and detached independently from the light-irradiation-side holding plate 102. The opening section 110 is desirably present in the light-irradiation-side-holding-plate supporting member 104 to make it easy to set and replace the light diffusing member 111.

Subsequently, preferred shapes and materials or characteristics of the members configuring the photoacoustic imaging apparatus are explained in detail.

(Light source)

The light source emits light having specific wavelength absorbed by a specific component (e.g., hemoglobin) among components configuring a living organism. Specifically, the light source desirably emits light having wavelength equal to or larger than 500 nm and equal to or smaller than 1200 nm. As the light source, at least one light source capable of generating pulsed light having pulse width of 5 nanoseconds to 50 nanoseconds is provided. As the light source, a laser that can obtain a large output is desirable. However, a light-emitting diode or the like can be used instead of the laser. As the laser, various lasers such as a solid-state laser, a gas laser, a dye laser, and a semiconductor layer can be used. An Nd:YAG laser and a Ti:sapphire laser can also be used. The laser may be variable wavelength.

(Optical system)

The optical system is, for example, a mirror that reflects light, a half mirror for splitting light into reference light and irradiation light, or a lens that condenses and magnifies light and changes the shape of the light. Examples of such an optical system include an optical waveguide besides the mirror or the lens. The optical system may be any optical system as long as light emitted from the light source can be irradiated on the object in a desired shape. It is preferable to expand the light to a certain degree of an area by diffusing the light with the lens.

A region where the light is irradiated on the object is desirably movable on the object. In other words, the optical system is configured such that the light emitted from the light source is movable on the object. Since the light is movable, it is possible to irradiate the light in a wide range. As a method of moving the region in which the light is irradiated on the object, there are, for example, a method of using a movable mirror or the like and a method of mechanically moving the light source itself.

(Light-amount measuring unit)

The light-amount measuring unit is a light power meter. As the light-amount measuring unit, there are, for example, an optical sensor that makes use of a photodiode, a thermal sensor that makes use of a thermocouple element, and a piroelectric electric sensor that makes use of a pyroelectric substance. Since the light source in the present invention has a single pulse, the pyroelectric sensor is desirable.

(Light diffusing member)

As the light diffusing member, for example, a thin urethane sheet including a scatterer, a light diffusing member, or titanium oxide is desirable. In isotropic circular diffusion, a diffusion angle is desirably uniform in a plane and sufficiently larger than an angle of view of the imaging unit and the lens. For example, when the angle of view is 20 degrees, a light diffusing member having a diffusion angle equal to or larger than 60 degrees is desirable to prevent the intensity of diffused light from changing in angles of ±10 degrees from the angle of view. The thickness of the light diffusing member is desirably about 0.1 mm to 1.0 mm. The light diffusing member is equivalent to a light diffusing unit according to the present invention.

(Imaging unit)

The imaging unit is a CCD camera or a CMOS camera. When combined with an optical system such as a lens or an ND filter, the imaging unit has focal length at which an entire optical pattern diffused on the light diffusing member 111 can be imaged. In order to calculate a light irradiation density distribution in the processing unit 107 on the basis of image data photographed by the photographing unit, it is desirable that the image data can be digitally output. The number of pixels needs to be a sufficient number taking into account an element pitch of the acoustic-wave converting unit 106 in an imaging region. In this embodiment, the imaging unit and the light diffusing member are equivalent to a light measuring unit.

(Acoustic-wave converting unit)

The acoustic-wave converting unit includes one or more elements that receive an acoustic wave and convert the acoustic wave into an electric signal. The acoustic-wave converting unit is configured by, for example, a transducer that makes use of a piezoelectric phenomenon, a transducer that makes use of resonance of light, or a transducer that makes use of a change in capacity. Any elements may be used as long as the elements can receive an acoustic wave and convert the acoustic wave into an electric signal. A plurality of the elements that receive an acoustic wave are one-dimensionally or two-dimensionally arrayed, whereby it is possible to simultaneously receive the acoustic wave in a plurality of places, reduce a reception time, and reduce the influence of, for example, vibration of the object. By moving one element, it is also possible to obtain a signal same as a signal obtained when the plurality of elements are one-dimensionally or two-dimensionally arranged. The acoustic-wave converting unit is equivalent to an acoustic receiving unit according to the present invention.

(Processing unit)

The processing unit 107 is a processing unit that performs, for example, calculation of a relative illuminance distribution, generation of image data such as a light absorption coefficient distribution, and command transmission to a focus adjusting mechanism. Typically, a workstation or the like is used. For example, processing for calculating a relative illuminance distribution and feeding back a result of the calculation to illumination light is performed by software programmed in advance. The processing unit may apply noise reduction processing or the like to an electric signal captured from the acoustic-wave converting unit 106.

Further, the processing unit 107 may perform not only movement control for the imaging unit 112 but also overall processing for causing the photoacoustic imaging apparatus to operate such as control of a scanning mechanism for the acoustic-wave converting unit 106, the optical system 109, and the like.

(Flow of overall light irradiation density distribution calculation processing)

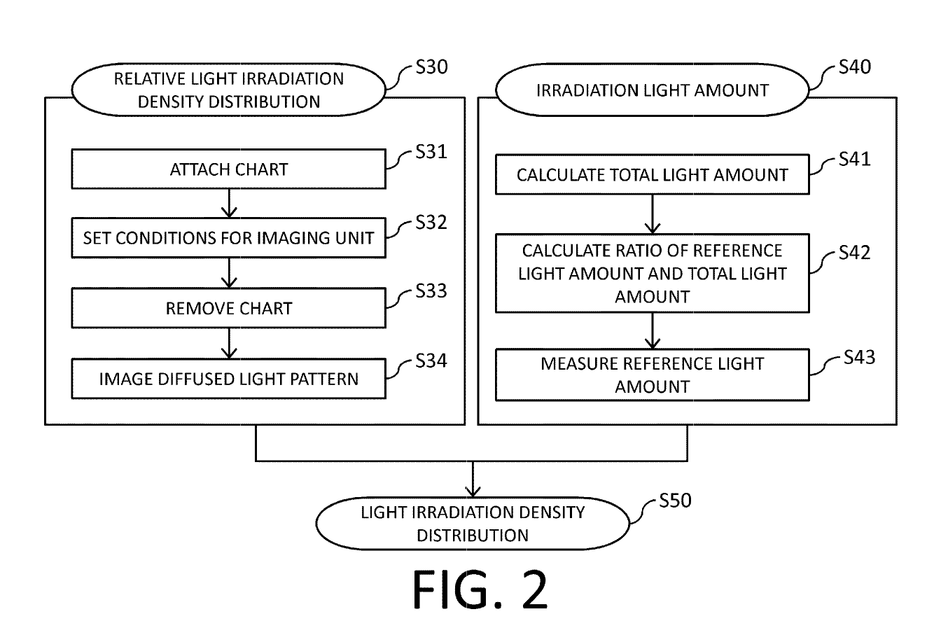

A workflow of this embodiment is explained with reference to FIG. 2.

In this work, acquisition of a relative light irradiation density distribution in step S30 and acquisition of an irradiation light amount in step S40 are performed. A light irradiation density distribution is calculated in step S50 on the basis of the relative light irradiation density distribution and the irradiation light amount.

(Acquisition method and mechanism for a relative light irradiation density distribution)

An acquisition method and a mechanism for a relative light irradiation density distribution (a relative illuminance distribution) are explained. A method used in PAT measurement for the acquired relative light irradiation density distribution is explained below. First, the configuration of an acquiring mechanism for a relative light irradiation density distribution, which is a characteristic of this embodiment, is explained.

A relative light irradiation density distribution means a relative light intensity distribution in a light irradiation region on the surface of the object 105. In this embodiment, when a relative light irradiation density distribution is acquired, as shown in FIG. 1B, the irradiation light 115 is irradiated on the light diffusing member 111 from the optical system 109. An irradiated optical pattern (diffused light pattern) is imaged by the imaging unit 112 and subjected to analysis processing by the processing unit 107, whereby a relative light irradiation density distribution is acquired.

In the flow of FIG. 2 explained above, sub-steps S31 to S34 are included in step S30 related to the acquisition of a relative light irradiation density distribution.

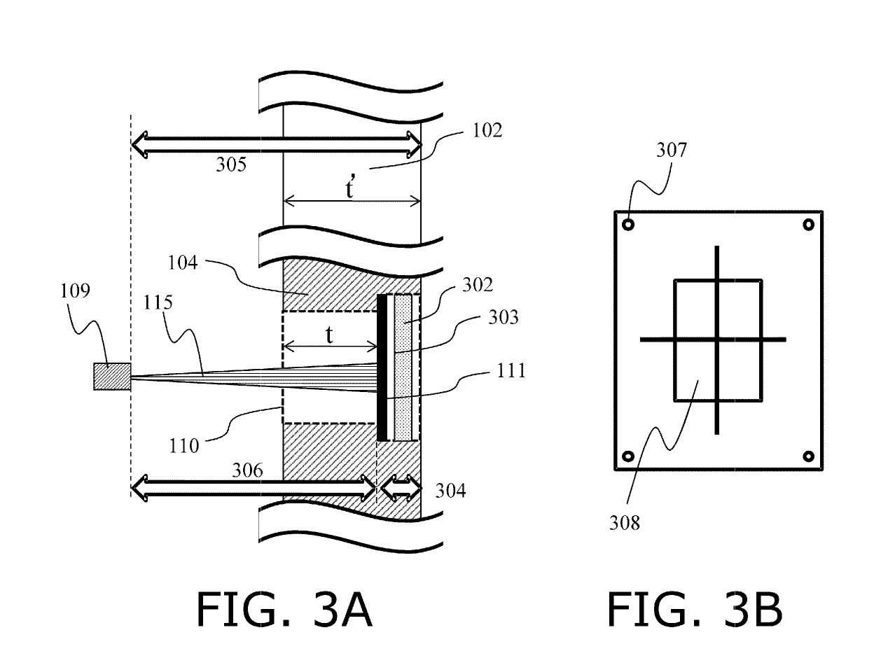

(Step S31) For accurate light measurement, it is necessary to adjust an angle of view and a focus of the imaging unit 112 to the light diffusing member 111 (FIG. 1B) fixed to the light-irradiation-side-holding-plate supporting member 104. Therefore, a chart or the like with which an angle of view and a focus can be determined is attached to the imaging unit side of the light diffusing member 111. The chart is attached as indicated by reference numeral 302 in FIG. 3A.

(Step S32) Subsequently, condition setting for imaging by the imaging unit 112 is performed. That is, the focus and the angle of view of the imaging unit 112 are adjusted according to an optical pattern (a diffused light pattern) irradiated on the surface of the light diffusing member 111 from the optical system 109.

(Step S33) Thereafter, the chart is removed again.

(Step S34) The diffused light pattern is imaged by the imaging unit 112 in a state in which the chart is removed. An image is subjected to analysis processing by the processing unit 107, whereby relative light irradiation density is calculated.

Work for the focus and image adjustment (S32) does not always need to be performed every time as long as the position of a diffuser does not change. However, when it is likely that the diffuser is replaced or the position of the diffuser is changed, it is necessary to perform the work every time the diffuser is replaced or the position of the diffuser is changed. Therefore, it is desirable that the light-irradiation-side holding plate 102 and the diffuser can be separately removed and the position of the diffuser does not change when the light-irradiation-side holding plate 102 is replaced.

(Attachment position of the light diffusing member)

Japanese Patent Application Laid-Open No. 2011-229756 describes that the light diffusing member is arranged on a surface on the side in contact with the object of the holding unit on the side where light is irradiated on the object or on substantially the same surface as the surface. However, when deflection occurs in the holding plate because of stress generated when the object is pressured, a distance of the irradiation light reaching the surface of the object sometimes changes (decreases). In that case, it is likely that a light irradiation amount based on an image imaged by arranging the diffusing member on the surface of the holding plate is inaccurate. Therefore, a preferable method of attaching the light diffusing member for coping with such a change in the distance is explained.

In FIG. 3A, when the light diffusing member 111 is fixed to the light-irradiation-side-holding-plate supporting member 104, a fixed distance 304 is kept between the light diffusing member 111 and the surface of the light-irradiation-side holding plate 102 that is in contact with the object 105. Consequently, it is possible to calculate an accurate relative light irradiation density distribution corresponding to the position of the light-irradiation-side holding plate 102 during actual photoacoustic measurement.

A suitable numerical value of the fixed distance 304 is desirably set to reduce an optical path difference between an optical path 305 from the optical system 109 to the object 105 and an optical path 306 from the optical system 109 to the light diffusing plate 111 to zero. For example, it is assumed that only the light-irradiation-side holding plate 102 is present between the optical system 109 and the object 105 except the air and only the air is present between the optical system 109 and the light diffusing member 111. In this case, taking into account a difference between refractive indexes of the atmosphere and the light-irradiation-side holding plate 102, when the refractive index of the air is represented as n, the refractive index of the light-irradiation-side holding plate 102 is represented as n′, and the thickness of the light-irradiation-side holding plate 102 is represented as t′, the fixed distance 304 can be represented as (1−n/n′)t′.

(Attachment of a chart)

An example of a chart pattern 308 is explained with reference to FIG. 3B. A cross clearly showing the center of a diffused light pattern and a shape for clearly showing the external shape (a square) of an illumination region are adopted as shown in the figure. Consequently, it is easy to calculate a size per one pixel.

An example of chart attachment is explained with reference to FIGS. 3A and 3B. Accuracy of focusing is higher when a pattern surface 303 of a chart 302 shown in FIG. 3A is attached to face the light diffusing member side. Therefore, for example, it is desirable to provide a protrusion shape in the chart 302 to form a shape (not shown) for preventing misattachment with wrong side out.

When the chart 302 deviates to the object side, replacement workability is improved. Therefore, for example, a step for positioning and attachment for the light diffusing member 111 and the chart 302 may be provided in the light-irradiation-side-holding-plate supporting member 104. The chart 302 may be fixed by screws through attachment holes 307 (FIG. 3B). A structure for clamping the chart 302 may be attached to the light-irradiation-side-holding-plate supporting member 104. Further, a cover glass (not shown in the figure) for prevention of stains of the light diffusing member 111 after the removal of the chart 302 may be attached.

In the imaging unit 112, an optical system (not shown) such as an ND filter or a lens is arranged. The lens desirably has a focal length at which the imaging unit 112 can image an entire optical pattern of the irradiation light 115 diffused on the light diffusing member 111. The ND filter desirably has ND at which the imaging unit 112 can image an entire optical pattern of the irradiation light 115 diffused on the light diffusing member 111.

(Acquisition of an irradiation light amount)

Referring back to the flow shown in FIG. 2, a light amount distribution in the object 105 is calculated using a light irradiation density distribution of light irradiated on the surface of the object 105. An example of a method of calculating a total light amount of the irradiation light 115 is explained. As well as the relative light irradiation density distribution (calculated in step S30 as explained above), the total light amount of the irradiation light 115 is necessary to calculate a light irradiation density distribution in S50, which is information concerning absolute intensity of light.

In the flow shown in FIG. 2, sub-steps S41 to S43 is included in step S40 related to acquisition of an irradiation light amount. The irradiation light amount is a total light amount of the irradiation light 115 irradiated on the light irradiation region on the surface of the object 105.

(step S41) In FIG. 1A, the optical system 109 includes an optical system (e.g., a half mirror) that splits light into the irradiation light 115 irradiated on the holding unit 101 and the reference light 116 irradiated on the light-amount measuring unit 113 that measures an amount of the light. In this step, in such a state, a total light amount is measured by a light power meter or the like in advance.

(Step S42) Subsequently, a ratio of a light amount of the irradiation light 115 and a light amount of the reference light 116 is calculated. Since the ratio of the light amounts of the irradiation light 115 and the reference light 116 can be determined by adjusting the half mirror, the ratio is calculated according to a value of the adjustment of the half mirror.

(Step S43) Subsequently, the light amount of the reference light 116 is monitored by the light-amount measuring unit 113. A total light amount of the irradiation light 115 can be learned at any time from a monitored value and the ratio in the preceding step.

The light-amount measuring unit 113 is not always necessary. An optical system that transmits only a desired amount of light may be used as the optical system 109. A ratio set during manufacturing of the photoacoustic imaging apparatus may be used.

(Acquisition of a light irradiation density distribution)

Subsequently, in step S50, a light irradiation density distribution, which is information concerning absolute intensity of light on the surface of the object 105, is calculated on the basis of the relative light irradiation density distribution and the irradiation light amount.

(Measurement flow)

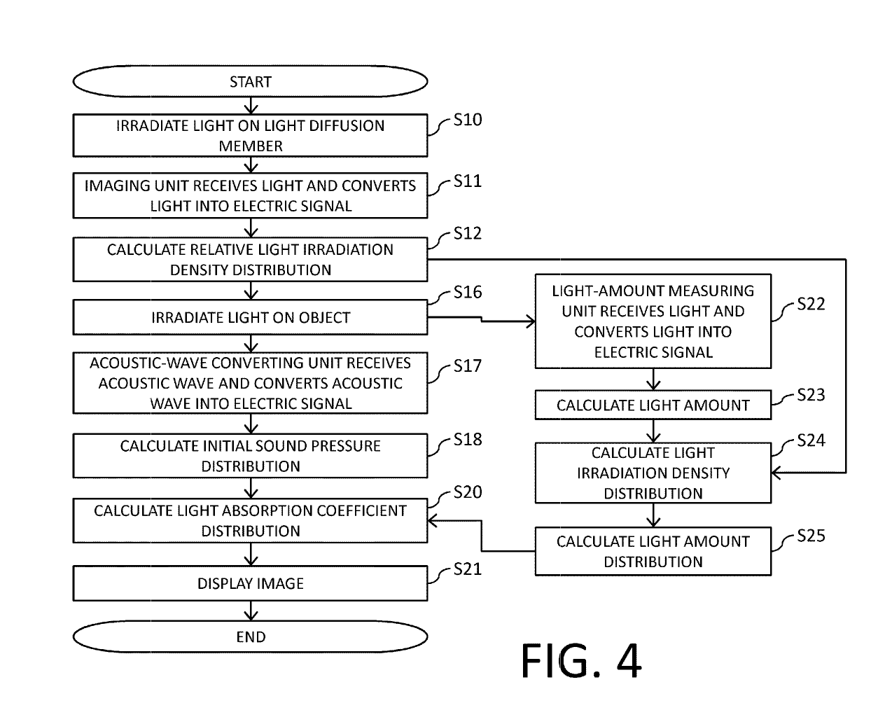

The operation of the photoacoustic imaging apparatus according to this embodiment is explained with reference to FIG. 4.

(Step S10) First, as shown in FIG. 1B, the light diffusing member 111 is arranged on substantially the same surface as a surface of the light-irradiation-side holding plate 102 that is in contact with the object 105. The light 114 from the light source 108 is irradiated on the light diffusing member 111 via the optical system 109 as the irradiation light 115.

(Step S11) The irradiation light 115 irradiated on the light diffusing member 111 is diffused on the light diffusing member 111. The imaging unit 112 images a diffused light pattern of the irradiation light 115 and converts the diffused light pattern into a first electric signal.

(Step S12) The first electric signal is captured into the processing unit 107. A relative light irradiation density distribution on the light diffusing member 111 is calculated. Specifically, the relative light irradiation density distribution is calculated on the basis of the diffused light pattern imaged by the imaging unit 112 and the size of an imaging target (the light diffusing member 111) per one pixel measured in advance.

(Step S16) Subsequently, as shown in FIG. 1A, the object 105 is held by the holding unit 101. In this way, the irradiation light 115 is irradiated on the object 105 in a state in which the acoustic-wave converting unit 106 and the irradiation light 115 are arranged in positions opposed to each other across the object 105.

(Step S17) The acoustic-wave converting unit 106 receives an acoustic wave generated when a part of optical energy of the irradiation light 115 is absorbed by the light absorber in the object 105 and converts the acoustic wave into a second electric signal.

(Step S18) The second electric signal is captured into the processing unit 107 and subjected to analysis processing by the processing unit 107 to calculate an initial sound pressure distribution.

(Step S22) On the other hand, a part of the light 114 branched from the irradiation light 115 irradiated on the subject 105 in step S16 is received by the light-amount measuring unit 113 as the reference light 116 and converted into an electric signal.

(Step S23) The electric signal after the converse is captured into the processing unit 107 and a light amount of the reference light 116 is calculated. A light amount of the reference light 116 and a light amount of the irradiation light 115 are adjusted to be a fixed ratio. Alternatively, the ratio is measured in advance. Therefore, the processing unit 107 can calculate a total light amount of the irradiation light 115 from the light amount of the reference light 116.

(Step S24) The processing unit 107 calculates a light irradiation density distribution on the surface of the object 105 from the total light amount of the irradiation light 115 (S23) and the relative light irradiation density distribution (S12).

(Step S25) The processing unit 107 calculates a light amount distribution in the object 105 on the basis of the light irradiation density distribution on the surface of the object 105.

(Step S20) The processing unit 107 calculates a light absorption coefficient distribution using the initial sound pressure distribution (S18) and the light amount distribution (S25).

(Step S21) Finally, the processing unit 107 generates, as image data, the light absorption coefficient distribution calculated in S20. The image data is displayed by a display device (not shown in the figure).

As explained above, a light irradiation density distribution is estimated using the light diffusing member 111. Therefore, it is possible to accurately calculate a light amount distribution of the object 105 and accurately calculate a light absorption coefficient distribution.

<Second Embodiment>

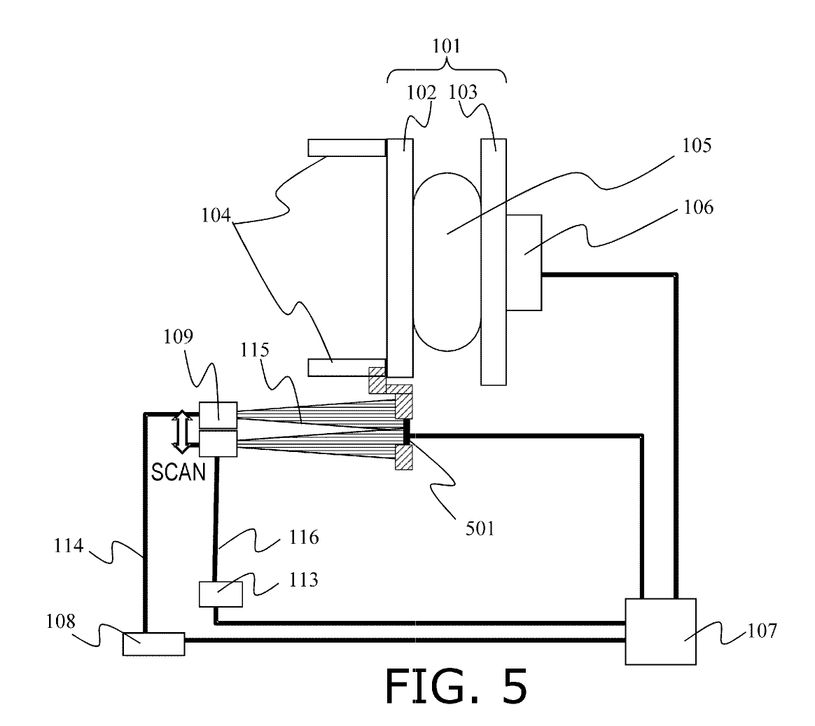

In the first embodiment, light is irradiated on the light diffusing member 111, an optical pattern generated in the light diffusing member 111 is imaged by the imaging unit 112 to calculate a relative light density distribution, and a total light amount is separately measured to calculate a light irradiation density distribution. In a second embodiment, instead of the light diffusing member 111 in the first embodiment, an irradiation light amount measurement sensor (reference numeral 501 in FIG. 5) is attached to the light-irradiation-side-holding-plate supporting member 104. With such a configuration, it is possible to simultaneously measure light intensity and distribution and directly calculate a light irradiation density distribution.

The irradiation light amount measurement sensor 501 includes one or more elements that detect light and convert the light into an electric signal. The irradiation light amount measurement sensor 501 is configured by a photomultiplier, a photodiode, or the like. Any elements may be used as long as the elements can detect light and convert the light into an electric signal. A plurality of the elements that detect light are one-dimensionally or two-dimensionally arrayed, whereby it is possible to simultaneously detect the light in a plurality of places and reduce a detection time. In this embodiment, the irradiation light amount measurement sensor 501 is equivalent to the light measuring unit. The irradiation light amount measurement sensor 501 can measure an amount of light emitted from a light source.

When the area of the irradiation light amount measurement sensor 501 is equal to or larger than the area of irradiation light, a light irradiation density distribution can be measured in one measurement. However, when the area of the irradiation light amount measurement sensor 501 is smaller than the area of the irradiation light, since a light irradiation density distribution cannot be measured in one measurement, the optical system 109 is scanned. Consequently, it is possible to measure a light irradiation density distribution of the entire region of an irradiation pattern.

When the irradiation light amount sensor explained in this embodiment is used, since a sensor wire is present, removal of the irradiation light amount sensor is more difficult than removal of the light diffusing member. Therefore, it is significant that replacement convenience is improved because the light-irradiation-side holding plate can be separately removed.

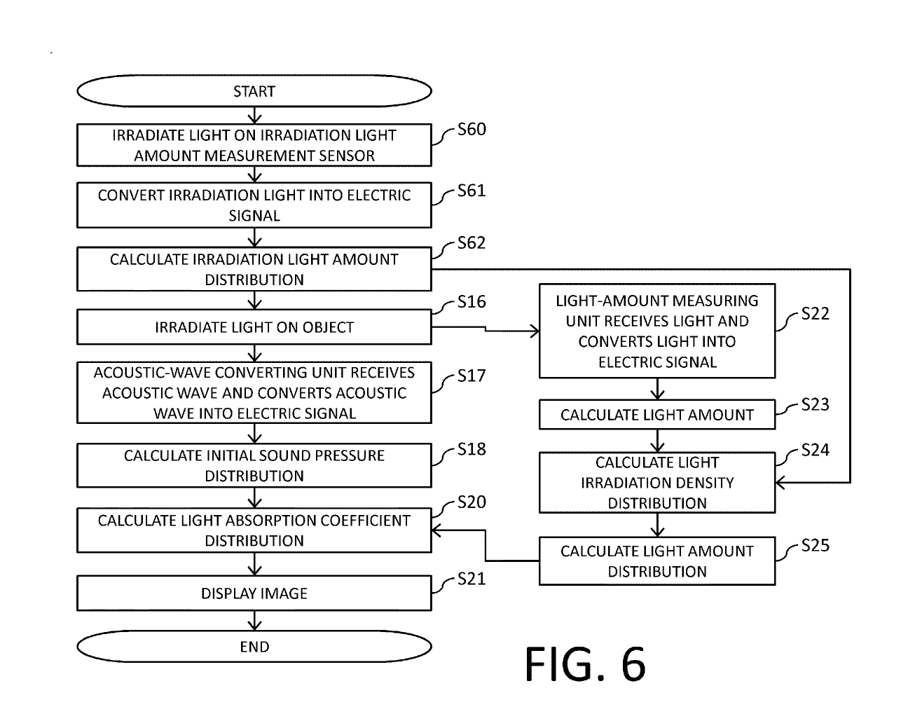

The operation of a photoacoustic imaging apparatus according to the second embodiment is explained with reference to FIG. 6.

(Step S60) First, as shown in FIG. 5, the irradiation-light-amount measurement sensor 501 is arranged on the substantially the same surface as a surface of the light-irradiation-side holding plate 102 that is in contact with the object 105. The light 114 from the light source 108 is irradiated on the irradiation light amount measurement sensor 501 via the optical system 109 as the irradiation light 115.

(Step S61) The irradiation light 115 irradiated on the irradiation light amount measurement sensor 501 is converted into a first electric signal. When the irradiation light 115 cannot be entirely measured by the irradiation light amount measurement sensor 501 at a time, the optical system 109 is scanned. Consequently, it is possible to obtain information concerning both a light amount and a distribution of the entire irradiation light 115.

(Step S62) The first electric signal is captured into the processing unit 107. An irradiation light amount distribution is calculated. In calculating the irradiation light amount distribution, it is possible to calculate irradiation density of light on the object 105 (S24) according to the intensity of reference light measured by the light-amount measuring unit 113 and the intensity of the reference light at the time when the light is irradiated on the object 105 (S16) and measured.

The operation after the irradiation of the light on the object 105 (S16) until the image display (S21) is the same as the operation in the first embodiment.

The light irradiation density distribution is calculated using the irradiation light amount measurement sensor 501 in this way. Consequently, it is possible to easily perform the calculation of a light irradiation density distribution using the light diffusing member 111. Since a light amount is directly measured by the sensor, it can be expected that a more accurate light irradiation density distribution can be calculated.

As explained above, in the object information acquiring apparatuses (the photoacoustic imaging apparatuses) according to the embodiments of the present invention, it is possible to independently replace the holding unit without removing the light measuring unit such as the light diffusing member. Consequently, it is possible to photograph the object held by the holding unit having the shape of the object and the shape matching an imaging purpose. Therefore, an image suitable for diagnosis can be easily obtained. Since the holding plate can be formed in a small and simple shape, it is possible to improve replacement easiness of the holding plate and reduce labor and time of an operator. The object information acquiring apparatuses are advantageous in terms of costs as well. Further, it is possible to more accurately and simply perform light irradiation density distribution measurement. Moreover, replacement easiness of the light measuring unit can also be obtained.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-051617, filed on Mar. 8, 2012, which is hereby incorporated by reference herein its entirety.

What is claimed is:

1. An object information acquiring apparatus comprising:
a light source;
a holding unit configured to hold an object;
an acoustic receiving unit configured to receive an acoustic wave generated by irradiating light, which is emitted from the light source, on the object via the holding unit and convert the acoustic wave into an electric signal;
a light measuring unit configured to measure the light emitted from the light source;
a processing unit configured to calculate a light density distribution in the object based on the light measured by the light measuring unit, and generate property information of an inside of the object on the basis of the light density distribution and the electric signal; and
a supporting unit configured to support the holding unit and at least a part of the light measuring unit to be removable independently from each other.

2. The object information acquiring apparatus according to claim 1, wherein
the light measuring unit includes:
a light diffusing unit attached to the supporting unit; and
an imaging unit configured to image an optical pattern generated when the light is irradiated on the light diffusing unit, and
the processing unit is configured to calculate the light density distribution on the basis of the optical pattern.

3. The object information acquiring apparatus according to claim 2, wherein
the supporting unit is configured to support the light diffusing unit.

4. The object information acquiring apparatus according to claim 1, wherein
the light measuring unit is a sensor configured to measure an emitted light amount from the light source, and
the processing unit is configured to calculate the light density distribution on the basis of the emitted light amount.

5. The object information acquiring apparatus according to claim 1, wherein
the holding unit is formed of a pair of plate-like members configured to sandwich the object, and
the supporting unit is configured to support one of the pair of plate-like members.
